(19) United States
(12) Patent Application Publication
(10) Pub. No.: US 2012/0041306 A1
(43) Pub. Date: Feb. 16, 2012

(54) CONTRAST AGENT ADMINISTRATION METHOD AND SYSTEM TO MAINTAIN CONTRAST AGENT AT A LOCALIZED SITE OF DELIVERY

(76) Inventors: Patrick Gross, Langensendelbach (DE); Rares Salomir, Ambilly (FR); Sylvain Terraz, Confignon (CH); Magalie Viallon, Lyon (FR); Anke Weissenborn, Weil am Rhein (DE)

(21) Appl. No.: 12/855,157

(22) Filed: Aug. 12, 2010

(51) Int. Cl. A61M 25/00; A61N 5/00; A61N 7/00; A61B 6/00; A61N 1/00

(52) U.S. Cl. 600/433; 600/431; 604/500; 604/20; 604/22; 604/523

(57) ABSTRACT

In a method and system for administering a dispersible medical agent to a subject, a volume of medical agent is delivered in vivo to a subject in a substantially non-dispersed state, at a localized site in tissue of the subject. Contemporaneously with administration of the medical agent to the localized site, energy from an energy source is introduced into only adjacent tissue surrounding the localized site. The energy produces a cellular modification of the adjacent tissue that makes the adjacent tissue substantially impermeable to passage of the medical agent therethrough, so the medical agent is encapsulated and is prevented from dispersing into tissue outside of the encapsulation.

### BACKGROUND OF THE INVENTION

1. Field of the Invention

The present invention concerns a method and a system to administer contrast agent, and in particular to a method and a system to maintain contrast agent at a localized site in the body of a subject after delivery of the contrast agent.

2. Description of the Prior Art

In many medical procedures and examinations, it is often desirable to fix an amount of liquid contrast agent or a therapeutic agent to a localized (small) volume or location within the anatomy of a subject, so that the contrast agent or the therapeutic agent will remain at the localized site for a time of sufficient length in the context of the particular examination or procedure that is being undertaken. This time may be several hours, days or even weeks. Systemically applied contrast agents and therapeutic agents, even if locally injected into a subject, will typically wash out or diffuse out of the site of delivery or administration after a relatively short time, or will be metabolized.

At present, the only manner to prevent such diffusion or metabolization of an administered contrast agent or a therapeutic agent is to use a solid volume of contrast agent or therapeutic agent, or to place pre-encapsulated liquid agents at the desired anatomical location. This can be appropriate, for example, if the administered agent is a contrast agent that is intended for use as a marker. In this case, however, it is not possible to administer or deliver the agents systemically. Moreover, such solid markers or capsules may not be compatible with the particular diagnostic imaging modality that will be used in the diagnosis or treatment plan of the subject. For example, metal markers may cause undesirable artifacts in magnetic resonance imaging.

### SUMMARY OF THE INVENTION

In accordance with the present invention, a systemically introduced or delivered contrast agent or a therapeutic agent, that is delivered in liquid form, is administered at a small, localized site in vivo in an examination subject. The administration of the fluid agent can be undertaken by means of a catheter, for example. Simultaneously or contemporaneously with the administration of the agent to the localized site, or immediately thereafter, energy is artificially introduced into tissue immediately surrounding (adjacent) the localized site at which the contrast agent is administered. The inflow of energy is produced in a manner and at a sufficient level to cause a cellular modification of the tissue surrounding the administered contrast agent, thereby encapsulating the contrast agent at that site, and substantially precluding diffusion, washing out, or metabolization of the administered contrast agent. The artificially applied energy may produce one or more of cell death, coagulation, absorption of the administered agent by the surrounding cells due to membrane disruption, or a ceasing of local metabolism. As a result of this cellular change, the administered agent is encapsulated either inside of cells or in a “shell,” with limited profusion, diffusion and/or metabolization.

The method and system in accordance with the present invention offer the advantages of a high degree of flexibility in the manner and type of contrast agent and therapeutic agent that can be administered and confined, a lower cost than conventional procedures for implanting solid markers or pre-encapsulated agents, as well as the ability to administer the contrast agent systemically. Moreover, the encapsulation that is created by the energy may have fewer and less significant side effects (i.e., be more biocompatible) compared to solid markers and pre-encapsulated agents. Moreover, the encapsulation produced by the cellular modification may be non-permanent so that after a length of time during which the contrast agent or the therapeutic agent has served its purpose, the encapsulation may disintegrate due to natural body processes, allowing the administered agent to slowly be diffused and absorbed in the body.

Whereas a solid contrast agent or a solid marker produce a strong local reflection of a HIFU beam, and subsequently a strong local heating and strong distortion of the beam, the encapsulated marker has little or no interaction with a HIFU beam. Therefore it is practically neutral with respect to a HIFU treatment that would follow its administration.

### DESCRIPTION OF THE DRAWINGS

FIG. 1 is a flow chart showing the basic steps of the method in accordance with the present invention.

FIG. 2 schematically illustrates a first embodiment of a system in accordance with the present invention.

FIG. 3 schematically illustrates a second embodiment of a system in accordance with the present invention.

FIGS. 4A and 4B schematically illustrate a third embodiment of a system in accordance with the present invention, FIG. 4A being a section taken through FIG. 4B.

### DESCRIPTION OF THE PREFERRED EMBODIMENTS

The inventive method and system are explained below in the context of administration of contrast agent, but as noted above the invention is not limited to the administration or delivery of contrast agent, but can be used to administer or deliver any type of fluid agent for any necessary medical purpose.

The basic steps in the embodiment of the method shown in FIG. 1 include step A, wherein contrast agent is delivered in a localized manner to an in vivo site, usually an interstitial site. In step B, energy is introduced into the tissue that surrounds the delivered contrast agent, so as to cause a cellular modification of that tissue that creates an encapsulation of the contrast agent. As used herein, “encapsulation” means that the administered contrast agent is prevented from any substantial interaction with tissue outside of the encapsulation for a length of time that is suitable and appropriate for the purpose for which the agent was administered. The encapsulation need not be, and most likely will not be, permanent.

Contrast agents are often made visible using a particular imaging modality, and/or may be optically visual to humans performing surgery or in making post-surgical assessments. Contrast agents can be used to assist in visualizing diseased tissue or tumors, or a healthy structure that is at risk and must be delineated for surgery. Contrast agent can also be administered to identify diseased tissue or tumors or a tissue sample that is to be delineated for treatment or that needs to be correlated with resected or ex-vivo sections. Contrast agent can also be used for lymph node marking, since many lymph nodes may not be easy to discriminate during surgery. For this purpose, lymph nodes can be made visible by magnetic resonance imaging or by the use of positron emission tomography (PET), in which case a PET tracer may need to be fixed at a localized site. Moreover, contemporaneously administered magnetic resonance markers and PET tracers may be used to correlate magnetic resonance images and PET images with each other.

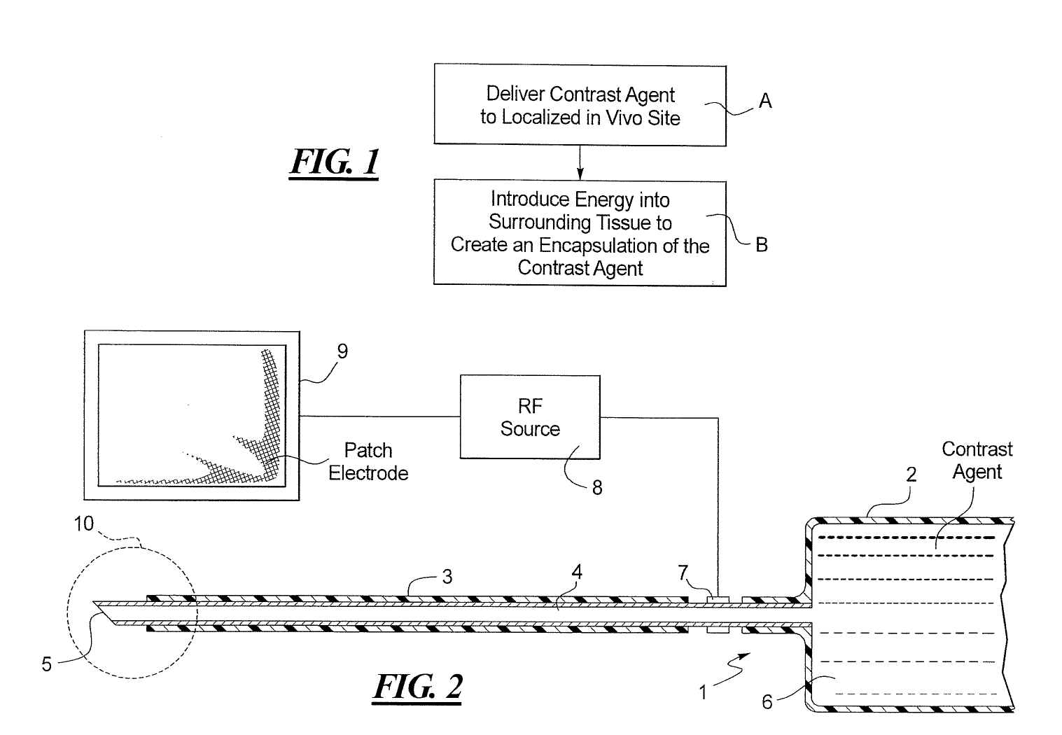

One embodiment of a system or implementing the method of FIG. 1 is shown in FIG. 2. This embodiment makes use of a schematically illustrated contrast agent injector 1, that has a chamber or reservoir 2 in which contrast agent is contained. The reservoir 2 is formed by a suitable non-conductive, substantially inert material, and this same, or a similar, material 3 extends around a catheter 4 that is in fluid communication with the interior of the reservoir 2. By any suitable application of pressure, the contrast agent contained in the reservoir 2 is forced out of the reservoir 2 through the catheter 4 and exits at the catheter tip 5. The catheter tip 5 will be placed in a suitable manner at an in vivo site at which the contrast agent is to be administered. The application of pressure is implemented in a manner so that only a small, localized volume of the contrast agent is expelled from the catheter tip 5 at the delivery site. The expelled volume thus, at least initially, is in a localized state.

In order to maintain the administered contrast agent in this localized state, in the embodiment of FIG. 2 RF energy from an RF source 8 is applied in order to cause a cellular modification of tissue in a region immediately surrounding the administered, localized contrast agent. The RF energy is administered in an RF circuit that includes the RF source 8, and a connection 7 to an exposed portion of the metal catheter 4. The connection 7 is shown in the form of a ring in the embodiment of FIG. 2, but it can be any suitable type of electrical connection, including a non-permanent connection, such as a clip or snap-on connection.

The RF circuit is completed by a patch electrode 9, that is extracorporeally placed at a location on the patient substantially opposite to the site at which the contrast agent is administered.

Upon activation of the RF source 8, the catheter tip 5 functions in the manner of an antenna, and creates an RF radiation pattern that is substantially spherical, and that surrounds the administered contrast agent, so as to produced the aforementioned cellular modification.

By suitably configuring the catheter tip 5, other radiation patterns can be achieved, as needed.

A further embodiment of a system for implementing the method of FIG. 1 is shown in FIG. 3, which makes use of a high intensity focused ultrasound (HIFU) source 11 as the source of cell-modifying energy. As is known, a HIFU source such as the HIFU source 11 has a HIFU focal region at which the focused ultrasound is effective. In the embodiment shown in FIG. 3, contrast agent is initially delivered to tissue at a localized site, and the HIFU source 11 is then activated after the administration. This is in contrast to the embodiment of FIG. 2, wherein the activation of the RF source 8 can ensue simultaneously with the delivery of the contrast agent. The HIFU source 11 causes a modification of the cells of tissue surrounding the delivered contrast agent within the HIFU focal region, thereby encapsulating the delivered contrast agent in the tissue.

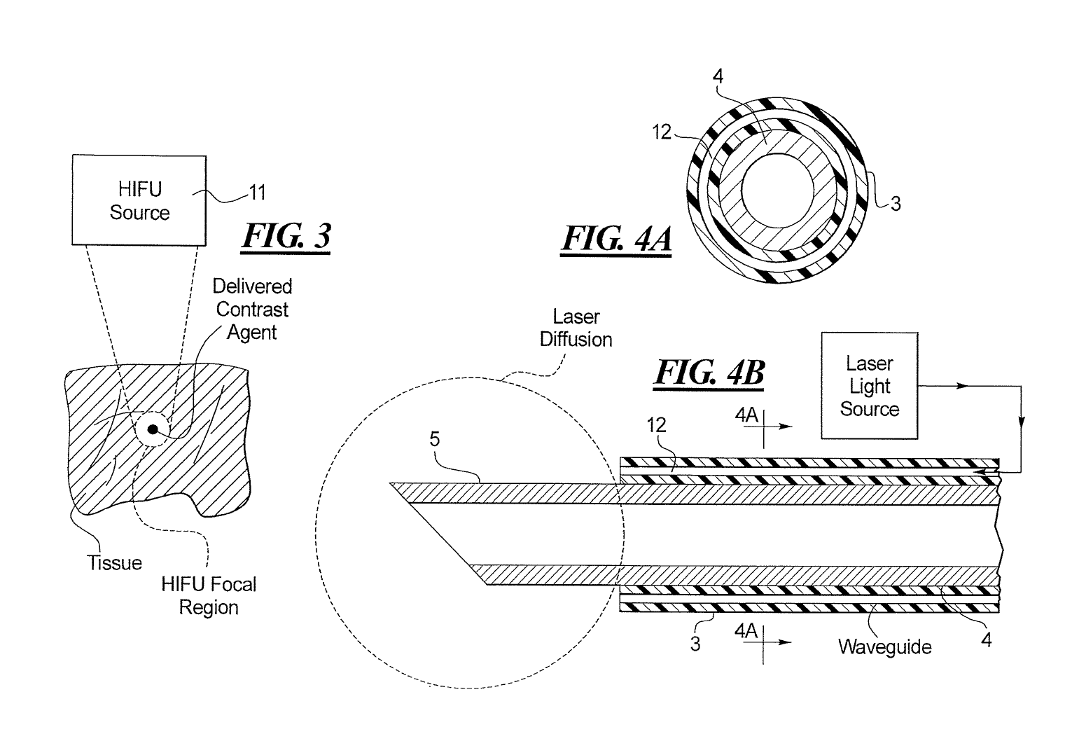

Another embodiment of a system in accordance with the present invention is shown in FIGS. 4A and 4B, which makes use of laser light as the source of energy for the cellular modification. FIG. 4B fills the distal end of a contrast agent injector that has a reservoir and other structure comparable to that shown in FIG. 2. In the embodiment of FIG. 4B, the catheter 4 is surrounded by an annular light waveguide 12, that conducts light from a laser light source to the catheter tip 5. Many types of structures and techniques for producing localized laser diffusion at the tip of a catheter are known, and any of these suitable structures and techniques can be employed in the embodiment of FIG. 4B to produce diffused laser light in a region or volume surrounding the catheter tip 5, so as to produce an encapsulation of the delivered contrast agent.

FIG. 4A shows the structure of FIG. 4B in section, taken along line 4A-4A of FIG. 4B.

As noted above, although the method and system have been described herein, in the context of administration of contrast agent, any type of agent can be administered in this manner, including mixtures of contrast agents and mixtures of contrast agents and therapeutic agents.

The systemic delivery of the contrast agent or therapeutic agent can also be done using micro-bubbles or coated structures. The encapsulating energy or another source of energy can be used to selectively release the agents at the target site, where the delivered agent has been fixed in location as described herein.

As is known, a suitable contrast agent that is visible in magnetic resonance imaging is a gadolinium-based contrast agent. This can be mixed with an optical agent so as to make the delivery visible to human perception at the delivery site as well.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for preventing in vivo dispersion of a dispersible medical agent, comprising the steps of:
delivering in vivo a volume of a dispersible medical agent, in a substantially non-dispersed state, to a localized site in tissue exhibiting tissue properties that tend to disperse said medical agent; and
contemporaneously with delivering said medical agent to said localized site, artificially introducing energy into substantially only adjacent tissue that surrounds said localized site, to produce a cellular modification of said adjacent tissue that makes said adjacent tissue substantially impermeable to passage of said medical agent therethrough, forming an encapsulation around said medical agent that maintains said medical agent at said localized site.

2. A method as claimed in claim 1 comprising delivering a volume of a substance, as said medical agent, selected from the group consisting of contrast agents and therapeutic agents.

3. A method as claimed in claim 1 comprising delivering said volume of said medical agent by interstitial injection of said volume of said medical agent into said tissue.

4. A method as claimed in claim 1 comprising producing said cellular modification of said adjacent tissue by irradiating said adjacent tissue with radio-frequency energy.

5. A method as claimed in claim 1 comprising producing said cellular modification of said adjacent tissue by exposing said adjacent tissue to laser light.

6. A method as claimed in claim 1 comprising producing said cellular modification of said adjacent tissue by irradiating said adjacent tissue with high intensity focused ultrasound.

7. A method as claimed in claim 1 comprising artificially introducing said energy into said adjacent tissue simultaneously with said delivery in vivo of said volume of medical agent.

8. A method as claimed in claim 1 comprising artificially introducing said energy into said adjacent tissue immediately following said in vivo delivery of said volume of said medical agent.

9. A system for administering a dispersible medical agent to a subject, comprising:
a source of a dispersible medical agent;
a delivery device in fluid communication with said source of said medical agent, said delivery device being configured to transfer a volume of said dispersible medical agent from said source and to deliver said volume of said dispersible medical agent in vivo, in a non-dispersed state, at a localized site in tissue of the subject; and
an energy source configured to introduce energy into substantially only adjacent tissue in the subject surrounding said localized site to produce a cellular modification of said adjacent tissue that makes said adjacent tissue substantially impermeable to passage of said medical agent therethrough, forming an encapsulation around the medical agent that contains said medical agent at said localized site.

10. A system as claimed in claim 9 wherein said delivery device is a catheter.

11. A system as claimed in claim 10 wherein said source of said medical agent is a reservoir in fluid communication with said catheter, said reservoir and said catheter forming an injector.

12. A system as claimed in claim 10 wherein said delivery device is a radio-frequency (RF) conductive catheter and wherein said energy source is an RF source in RF-conducting communication with said catheter, and wherein said system further comprises a patch electrode adapted for extracorporeal placement on the subject to complete an RF circuit with said RF source and said catheter.

13. A system as claimed in claim 9 wherein said energy source is a high intensity focused ultrasound source.

14. A system as claimed in claim 9 wherein said energy source is a source of laser light, and wherein said delivery device comprises a catheter surrounded by a light waveguide in optical communication with said source of laser light, said catheter terminating in a catheter tip at which said volume of medical agent is discharged from said catheter, and said light waveguide terminating adjacent said catheter tip to produce a field of dispersed laser light around said catheter tip.

15. A system as claimed in claim 9 wherein said delivery system, in addition to delivering said volume of said medical agent, comprises an energy delivery component in communication with said energy source that introduces said energy into said adjacent tissue to produce said cellular modification of said adjacent tissue.
